(19) United States
(12) Patent Application Publication
(10) Pub. No.: US 2013/0261673 A1
(43) Pub. Date: Oct. 3, 2013

(54) QUAD ANCHOR LATERAL VERTEBRAL BODY FIXATION PLATES

(71) Applicants: John Riley Hawkins, Cumberland, RI (US); Nicholas Pavento, North Attleboro, MA (US); Sean Selover, Westport, MA (US); Jonathan Bellas, Raynham, MA (US); Sheryl Frank, Lakeville, MA (US)

(72) Inventors: John Riley Hawkins, Cumberland, RI (US); Nicholas Pavento, North Attleboro, MA (US); Sean Selover, Westport, MA (US); Jonathan Bellas, Raynham, MA (US); Sheryl Frank, Lakeville, MA (US)

(21) Appl. No.: 13/432,321

(22) Filed: Mar. 28, 2012

(51) Int. Cl. A61B 17/80

(52) U.S. Cl. 606/286

(57) ABSTRACT

A bone plate secured by both a screw and a post. The head of the post may act as a cam that prevents screw backout. Preferably, the shaft of the first post passes partially through the first post through hole so that the distal unthreaded portion of its shaft substantially extends beyond the plate and the proximal threaded portion of its shaft substantially remains in the first post through hole.

### BACKGROUND OF THE INVENTION

A lateral access approach is frequently selected to deliver interbody fusion cages to the lumbar spine. In comparison with conventional anterior or posterior approaches to the lumbar spine, the lateral approach is thought to minimize posterior and/or anterior tissue damage as well as reduce surgery time, associated blood loss, vascular damage and infection risk.

In general, it is known in the art to mount a lateral fusion cage with a plate that secures the cage to the sides of adjacent vertebral bodies.

One problem associated with these designs is that the plate often has a greater width than the associated cage, thereby requiring the use of a larger diameter tube to pass them from the patient's skin to the patient's spine. This detracts from the goal of providing minimally invasive access to the spine.

U.S. Pat. No. 7,594,931 (Louis) discloses an intervertebral arthrodesis implant for insertion in an intervertebral space separating opposite faces of two adjacent vertebrae. The implant has a ring-shaped intervertebral cage having a bar that extends perpendicular to the axis of the spine. The bar has a height less than the rest of the cage. A surface of the cage contacting the vertebrae has an undulating shape for limiting sliding of the cage in a plane parallel to the vertebral faces.

PCT Published Patent Application WO2011-080535 (Dinville) discloses anchoring devices, anchoring systems for intervertebral implants, intervertebral implants, and instruments and methods for implanting the implants. In preferred configurations, these various objects share the feature of comprising or cooperating with an anchoring device having a body comprising at least one curved plate elongated along a longitudinal axis. The plate is designed to be inserted through a passage crossing at least a part of the implant in order to penetrate into at least one vertebral endplate and attach this implant onto this vertebral endplate by means of at least one stop retaining the implant. The body of the anchoring device comprises at least one longitudinal rib on at least a part of at least one of its faces, the rib being designed to cooperate with a groove made in a passage of implant.

In one type of intervertebral device suited for the lateral approach, the fusion cage is mounted with a plate that secures the cage to the adjacent vertebral bodies. In particular, US Published Patent Application 2010-0004747 (Lin) discloses a spinal fixation device comprising a trans-vertebral and intra-vertebral plate and a rectangular cage with a slot for the plate for neutralizing intervertebral movement in spinal interbody fusion. The rectangular cage with a vertical or oblique slot is inserted into the intervertebral space from the lateral or anterior side of the spinal column. The plate is then inserted through the slot of the cage and hammered into and buried inside the two adjacent vertebral bodies to achieve three-dimensional intervertebral fixation.

U.S. Pat. No. 8,002,808 (Morrison) discloses a device for supporting and/or assisting in bone fusion, particularly in the spine. A plate member is provided, along with two or more attachment members that are anchorable to bones. In one embodiment, the plate member has a slot near one end and an aperture at another end. The attachment members include threaded posts for connection to the plate member via the latter's slot(s) and aperture(s). Alternatively, attachment members need not have a threaded post, and attachment members may be connected to the plate member via a bone bolt or similar fixation member. The slot(s) allow a single plate member to be used for a variety of operative situations and anatomies. A device for repositioning bones and a method for using the disclosed devices is also described.

U.S. Pat. No. 7,985,223 (Khodadadyan-Klostermann) discloses a ventral bone-plate assembly has a U-shaped base having a pair of legs bridged by a bight and each formed with an inner hole and an outer hole. A brace bar extends between outer ends of the legs and is formed with end holes alignable with the outer holes. Respective inner bone screws extend through the inner holes, and respective outer bone screws each extend through a respective one of the outer holes and a respective one of the end holes.

PCT Published Application No. WO2004-098448 (RSB Spine LLC) discloses bone plate system comprising a base plate having two generally parallel elongated screw slots extending therethrough. Two bone screws are provided that are capable of securing the base plate to a bone by insertion through the screw slots into the bone. Each bone screw has a screw head and a threaded portion extending therefrom. An interference device is attached to the base plate and retains the bone screws while permitting the bone screws to toggle and to controllably slide in the screw slots of the base plate. This design is particularly useful for joining adjacent vertebral bodies, as it permits controlled settling of the vertebral bodies, thereby enhancing the healing process.

U.S. Pat. No. 7,341,590 (Ferree) discloses an anterior thoracic/lumbar system comprising a thin plate and fasteners for securing the plate to vertebrae or other osseous material. The plate may be hinged along the central axis, with a pair of collinear holes on each portion of the plate. Each of the holes accommodates a bolt which is screwed into the vertebrae and secured to the plate using a nut.

### SUMMARY OF THE INVENTION

One aspect of the present invention is directed to a bone plate secured in the bone by both a screw and a post. The shaft of the screw is threaded. The distal portion of the shaft of the post is unthreaded while the proximal portion thereof is threaded. The post has a head that may directly (as a cam) or indirectly (through a lip on a connector plate) prevent screw backout. Preferably, the distal portion of the shaft of the post passes through its hole in the bone plate so that the distal unthreaded portion of the shaft substantially extends beyond the bone plate and the proximal threaded portion of its shaft substantially remains in its (threaded) through hole. The advantage of this embodiment is that the post not only prevents screw backout, it also aids in plate securement to the bone.

In some embodiments, the post directly prevents backout of the bone screw from the first screw throughhole, whereby the head of the post bears against the head of the screw. In other embodiments, the bone post indirectly prevents backout of the bone screw from the first screw throughhole, whereby the head of the post bears upon an intermediate member (such as a connector plate) which in turn bears upon the head of the screw.

In a first preferred aspect of the present invention, the conventional set screw component of a cam lock (which does not penetrate the bone) is replaced with a second bone fixation anchor that penetrates the bone. As this second anchor is inserted into the plate and bone, its proximal head bears against the head of the first anchor, thereby performing the function of a cam lock while adding to the overall securement of the assembly in the bone. This also allows dynamization of the first anchor with the plate while preventing backout of the first anchor. Preferably, this second fixation anchor is a post whose bone-penetrating distal portion of the shaft is unthreaded and whose proximal portion of the shaft is threaded only to the extent that it mates with its correspondingly-threaded hole in the plate. In some embodiments, a pair of camming screw-and-post combinations are seated in a single bone plate, thereby providing design simplicity.

Therefore, in accordance with the present invention, there is provided a bone fixation system comprising:
a) a bone plate comprising:
   i) a first screw through hole having a first threadform,
   ii) a first post through hole having a second threadform having an axial length,
b) a first bone screw comprising a head and a shaft having a thread thereon that mates with the first threadform;
c) a first bone post comprising a head and a shaft having a thread thereon that mates with the second threadform and has an axial length;

wherein the first screw is received in the first screw through hole and extends therethrough,

wherein the first post is received in the first post through hole and extends therethrough,

wherein the head of the first post bears against the head of the first screw,

wherein the axial length of the thread of the first post is substantially the same as the axial length of the second threadform of the first post through hole.

In some camming embodiments, the pair of screw-and-post combinations are seated in separate bone plates that are linked together by a connecting plate, wherein the head of each post acts as a cam that prevents backout of its respective screw. This embodiment holds particular advantage in that each plate (i.e., the two bone plates and the connecting plate) may be designed to be relatively long and thin, thereby allowing for their individual, minimally-invasive deliveries to the spinal site through an MIS portal.

Therefore, in accordance with the present invention, there is provided bone fixation system comprising:
a) a first bone plate comprising:
   i) a first screw through hole having a first threadform,
   ii) a first post through hole having a second threadform having an axial length,
b) a first bone screw having a head and a shaft having a thread thereon that mates with the first threadform;
c) a first anchor having a head and a shaft having a thread thereon that mates with the second threadform and has an axial length;
d) a connector plate having a first throughhole and a second throughhole,
e) a second bone plate comprising:
   i) a second screw through hole having a third threadform,
   ii) a second anchor recess having a fourth threadform having an axial length,
f) a second bone screw having a head and a shaft having a thread thereon that mates with the third threadform;
g) a second anchor having a head and a shaft having a thread thereon that mates with the fourth threadform and has an axial length;
wherein the first screw is received in the first screw through hole and extends therethrough,
wherein the first anchor is received in the first anchor recess,
wherein the connector plate is located between the head of the first anchor and the first bone plate,
wherein the head of the first anchor bears against the head of the first screw,
wherein the axial length of the first anchor thread is substantially the same as the axial length of the second threadform.
wherein the second screw is received in the second screw through hole and extends therethrough,
wherein the second anchor is received in the second anchor recess,
wherein the connector plate is located between the head of the second post and the second bone plate,
wherein the head of the second anchor bears against the head of the second screw,
wherein the axial length of the second anchor thread is substantially the same as the axial length of the fourth threadform.

In a second preferred aspect of the present invention, the locking is performed by a snap tab. The first anchor snaps into the head of a staple portion with a spherical socket. The second anchor is placed and at the same time approximates a connecting plate to bridge to an adjacent vertebral bone staple. The connecting plate prevents the tab from un-snapping, which keeps the first anchor in a locked position.

Therefore, in accordance with the present invention, there is provided bone fixation system comprising:
a) a first bone plate comprising:
   i) a first screw through hole having a first threadform,
   ii) a first post through hole having a second threadform having an axial length,
   i) a first lip extending therefrom and
b) a first bone screw having a head and a shaft having a thread thereon that mates with the first threadform;
c) a first bone post having a head and a shaft having a thread thereon that mates with the second threadform and has an axial length;
d) a connector plate having a first throughhole,
wherein the first screw is received in the first screw through hole and extends therethrough,
wherein the first post is received in the first post through hole and the connector plate through hole, and extends therethrough,
wherein the connector plate is located between the head of the first post and the first bone plate,
wherein the first lip of the first bone plate bears against the head of the first screw,
wherein the connector plate bears against the first lip,
wherein the axial length of the thread of the first post is substantially the same as the axial length of the second threadform.

In preferred embodiments of this second aspect, the invention comprises a pair of screw-post-bone plate combinations linked together by a connecting plate that is fixed to each bone plate by the pair of posts. This embodiment holds particular advantage in that each plate (i.e., the two bone plates and the connecting plate) may be relatively long and thin, thereby allowing for their minimally invasive delivery to the spinal site through an MIS portal.

Therefore, in accordance with the present invention, there is provided bone fixation system comprising:
a) a first bone plate comprising:
   i) a first screw through hole having a first threadform,
   ii) a first post through hole having a second threadform having an axial length,
b) a first bone screw having a head and a shaft having a thread thereon that mates with the first threadform;
c) a first anchor having a head and a shaft having a thread thereon that mates with the second threadform and has an axial length;
d) a connector plate having a first lip extending therefrom and a first throughhole and a second lip extending therefrom and a second throughhole,
e) a second bone plate having a thickness and comprising:
   i) a second screw through hole having a third threadform,
   ii) a second anchor recess having a fourth threadform having an axial length,
f) a second bone screw having a head and a shaft having a thread thereon that mates with the third threadform;
g) a second anchor having a head and a shaft having a thread thereon that mates with the fourth threadform and has an axial length;
wherein the first screw is received in the first screw through hole and extends therethrough,
wherein the first anchor is received in the first anchor recess,
wherein the connector plate is located between the head of the first anchor and the first bone plate,
wherein the first lip of the connector plate bears against the head of the first screw,
wherein the axial length of the first anchor thread is substantially the same as the axial length of the second threadform,
wherein the second screw is received in the second screw through hole and extends therethrough,
wherein the second anchor is received in the second anchor recess,
wherein the connector plate is located between the head of the second post and the second bone plate,
wherein the second lip of the connector plate bears against the head of the second screw,
wherein the axial length of the second anchor thread is substantially the same as the axial length of the fourth threadform,

In a third aspect of the present invention, the head of the primary bone screw is cammed by the head of a secondary bone anchor having a machine thread. In this embodiment, the thread on the secondary bone anchor is more fine than the thread of the associated bone screw. The fineness of thread of the secondary bone anchor is an advantage because it provides a greater locking torque.

Therefore, in accordance with the present invention, there is provided bone fixation system comprising:
a) a first bone plate having a thickness and comprising:
   i) a bone screw through hole having a first threadform,
   ii) a bone anchor through hole having a second threadform,
b) a first bone screw having a head and a shaft having a thread thereon that mates with the first threadform;
c) a first bone anchor having a proximal head, an intermediate shaft and a distal tip, the shaft having a proximal threaded portion that mates with the second threadform,
wherein the first screw is received in the first screw through hole and extends therethrough,
wherein the first bone anchor is received in the first bone anchor through hole and extends therethrough,
wherein the head of the bone anchor bears against the head of the bone screw,
wherein the second threadform is more fine than the first threadform.

### DESCRIPTION OF THE FIGURES

FIGS. 1-4 disclose an embodiment of the present invention in which the head of the bone post cams the head of the bone screw.

FIGS. 5-8 disclose an embodiment of the present invention in which the head of a bone screw cams the head of a second bone screw.

FIGS. 9-11 disclose an embodiment of the present invention in which the overall plate comprises a pair of bone plates connected by a connector plate.

FIGS. 12-13 disclose the embodiment of FIGS. 9-11 but without the connector plate.

FIGS. 14-17 disclose an embodiment in which the camming function is performed by a snap-on plate.

### DETAILED DESCRIPTION OF THE INVENTION

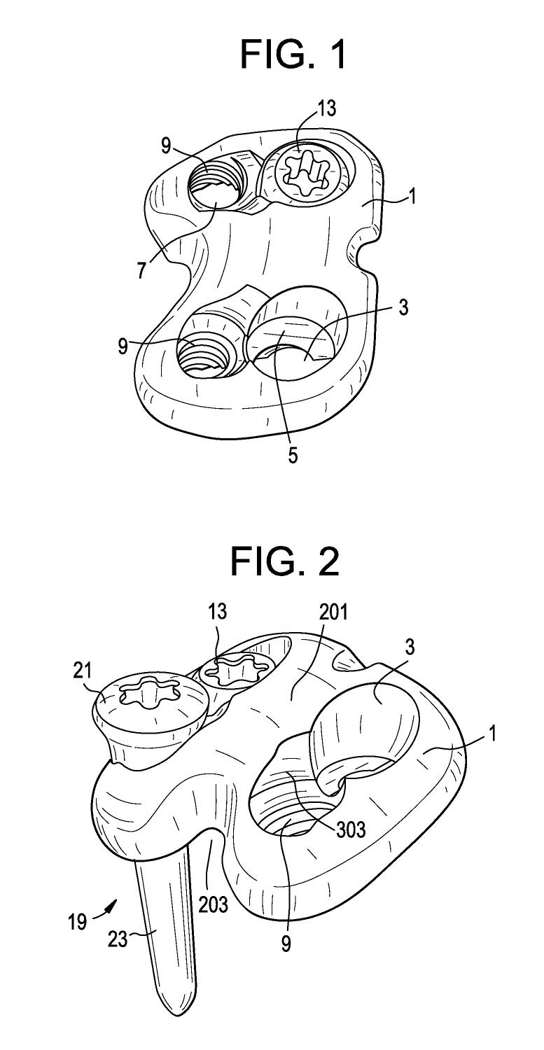

Now referring to FIGS. 1-4, there is provided a bone fixation system comprising:
a) a bone plate 1 having a thickness and comprising:
   i) first and second screw through holes 3 having a first threadform 5,
   ii) first and second post through holes 7 having a second threadform 9 having an axial length,
b) first and second bone screws 11, each screw having a head 13 and a shaft 15 having a thread 17 thereon that mates with the first threadform;
c) first and second bone posts 19, each post having a head 21 and a shaft 23 having a thread (not shown) thereon that mates with the second threadform, each thread having an axial length;
wherein each screw is received in a respective screw through hole and extends therethrough,
wherein each post is received in a respective post through hole and extends therethrough,
wherein the head of each post bears against the head of a respective screw,
wherein the axial length of each post thread is substantially the same as the axial length of its respective second threadform.

In this embodiment, the conventional cam is replaced by a bone post whose shaft has an unthreaded distal portion. Therefore, this embodiment provides additional securement of the plate to the bone without adding a “backout” failure mode.

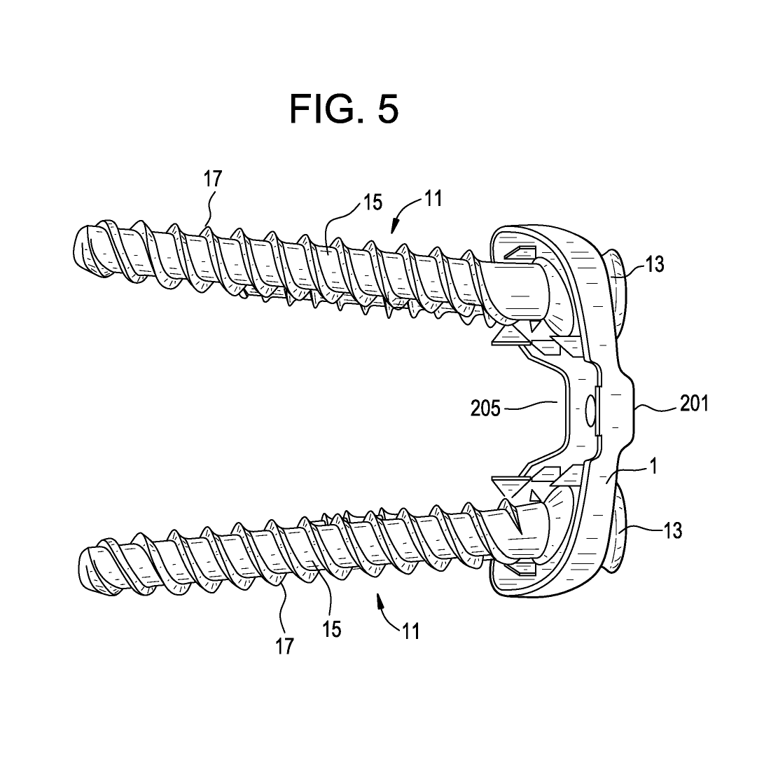

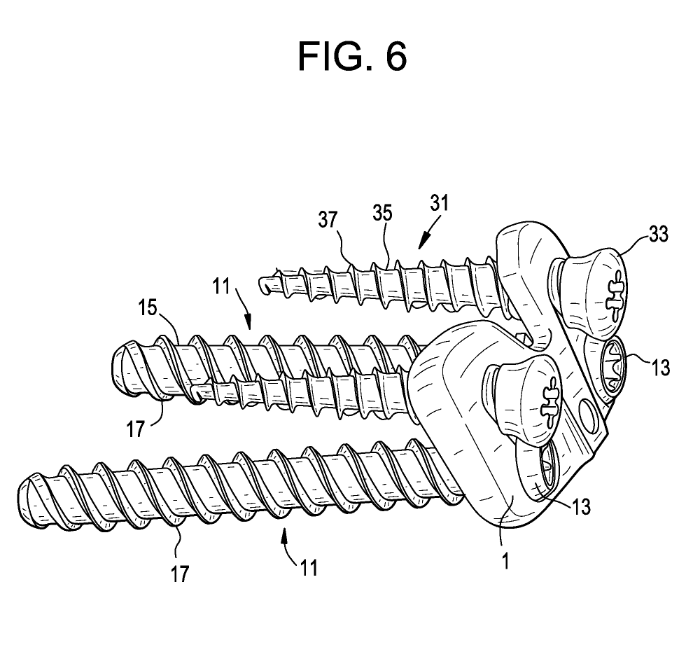

In FIGS. 5-8, the bone posts are replaced by bone screws 31, each screw having a head 33, and a shaft 35 having a thread 37 thereon.

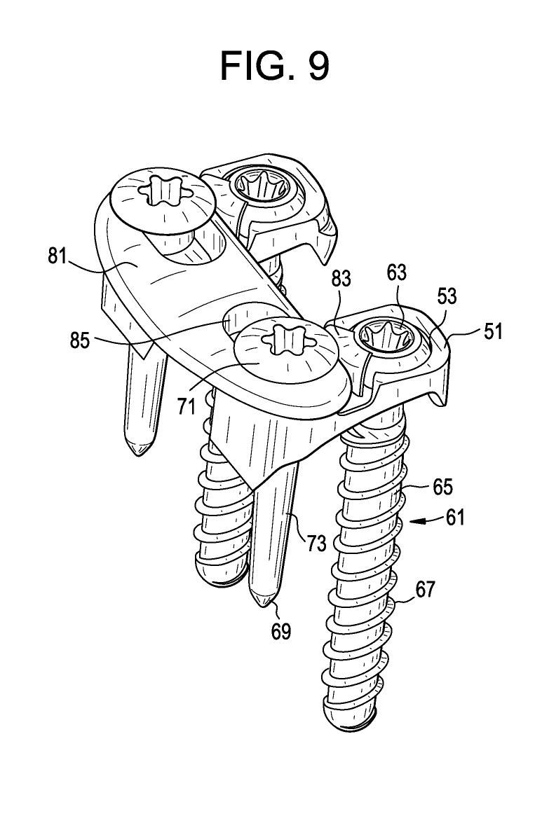

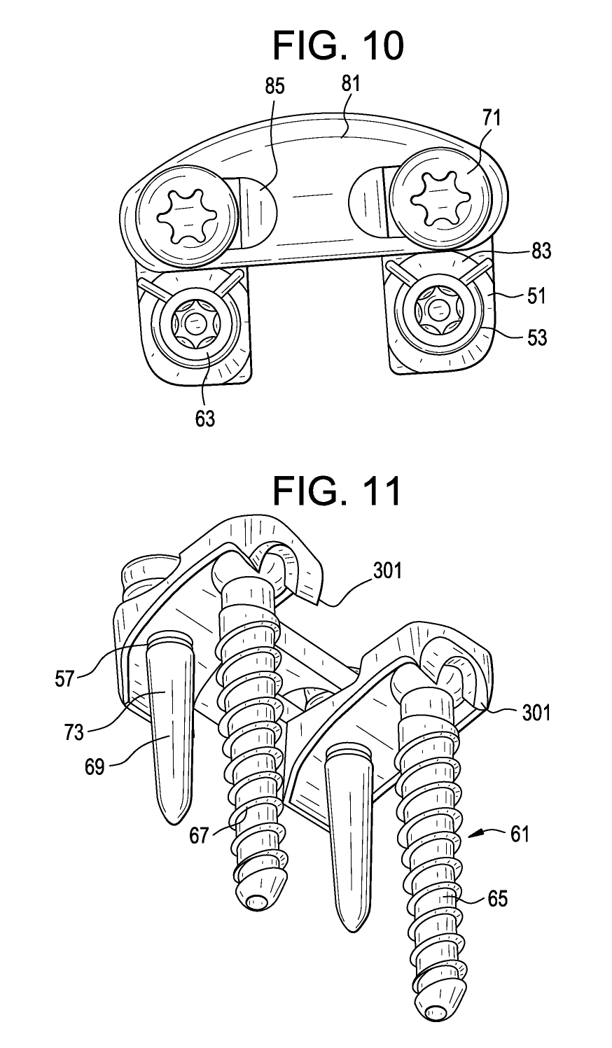

Now referring to FIGS. 9-13, there is provided a bone fixation system comprising:
a) a first bone plate 51 having a thickness and comprising:
   i) a first screw through hole 53 having a first threadform (not shown),
   ii) a first post through hole (not shown) having a second threadform (not shown) having an axial length,
   iii) a lip 83 extending therefrom,
b) a first bone screw 61 having a head 63 and a shaft 65 having a thread 67 thereon that mates with the first threadform;
c) a first bone post 69 having a head 71 and a shaft 73 having a thread (not shown) thereon that mates with the second threadform and has an axial length;
d) a connector plate 81 having a first throughhole 85,
wherein the first screw is received in the first screw through hole and extends therethrough,
wherein the first post is received in the first post through hole and the connector plate through hole, and extends therethrough,
wherein the connector plate is located between the head of the first post and the first bone plate,
wherein the lip of the first bone plate bears against the head of the first screw,
wherein the connector plate bears against the lip,
wherein the axial length of the first post thread is substantially the same as the axial length of the second threadform.

In FIGS. 9-11, the “overall” plate is divided into three subparts: the first plate, the connector plate and the second plate. In a first step of the surgical technique, the first and second plates are secured to the adjacent vertebral bodies by their respective bone screws. In a second step of the surgical technique, the connector plate is laid on top of the two staples. Bone anchors are then passed through the two overlapping portions of the staples and connector. The advantage of this embodiment is that the overall plate is split up into three relatively thin components, each of which having a width that is only slightly greater than the diameter of the associated bone anchors. Accordingly, these three relatively narrow plate components can be easily separately slid down the same MIS portal that passes the associated cage, and then assembled in-situ.

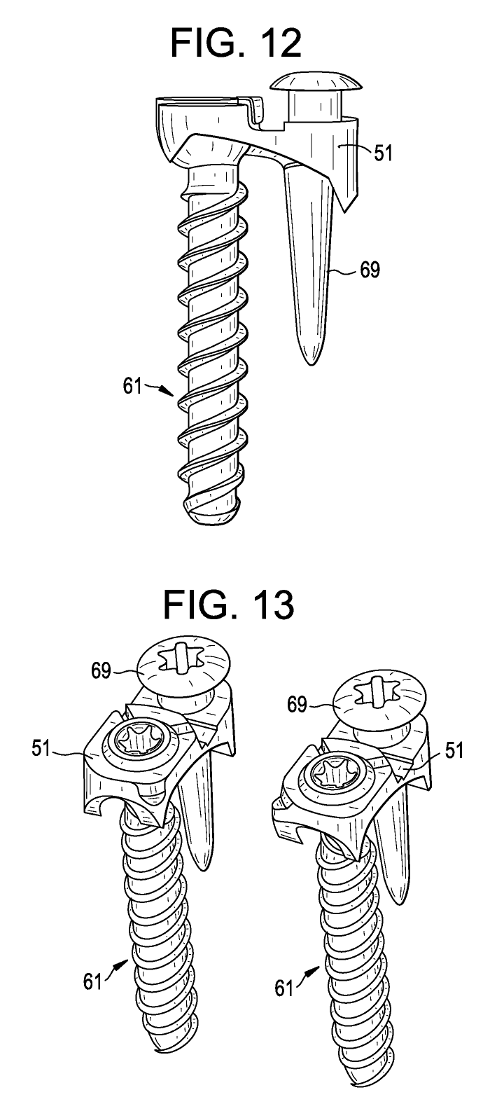

FIGS. 12-13 show versions of the embodiment of FIGS. 9-11 but without the connector plate.

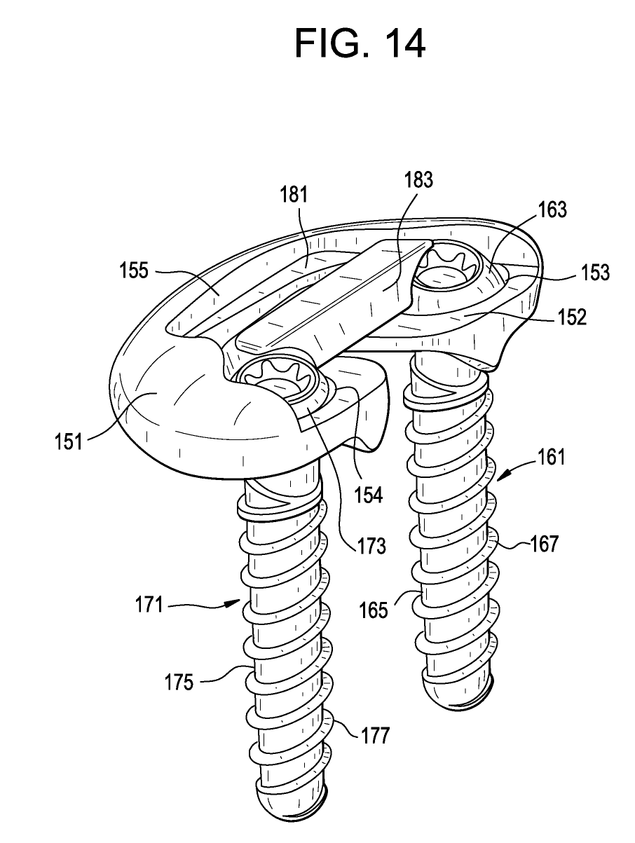

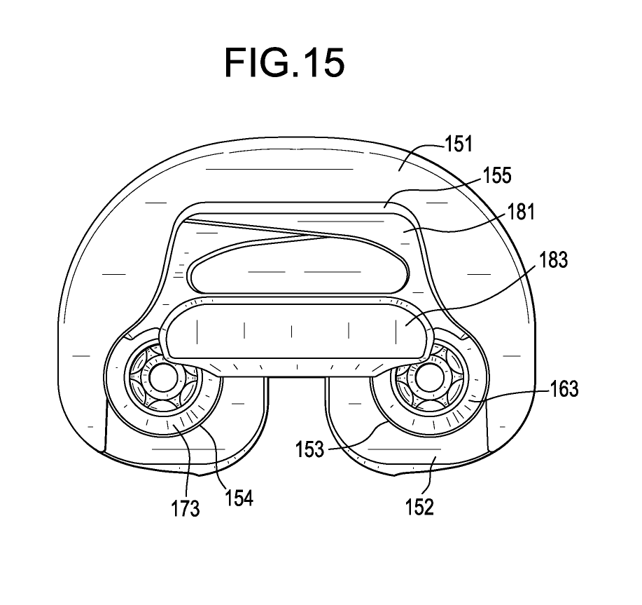

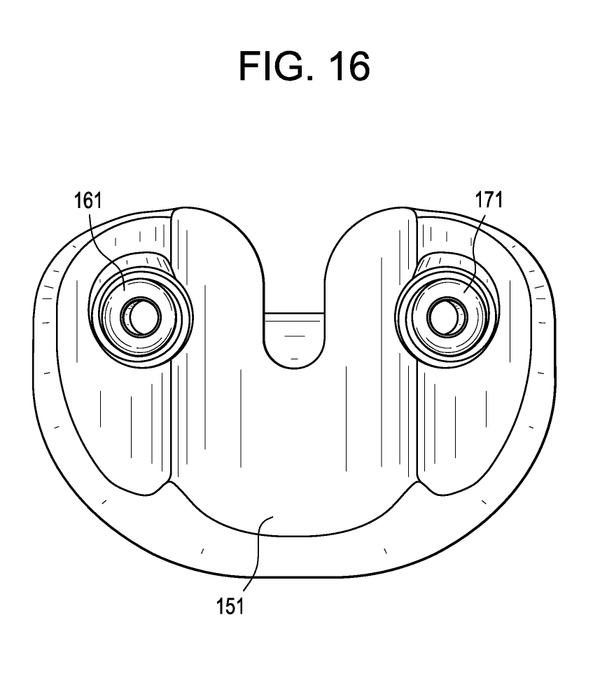

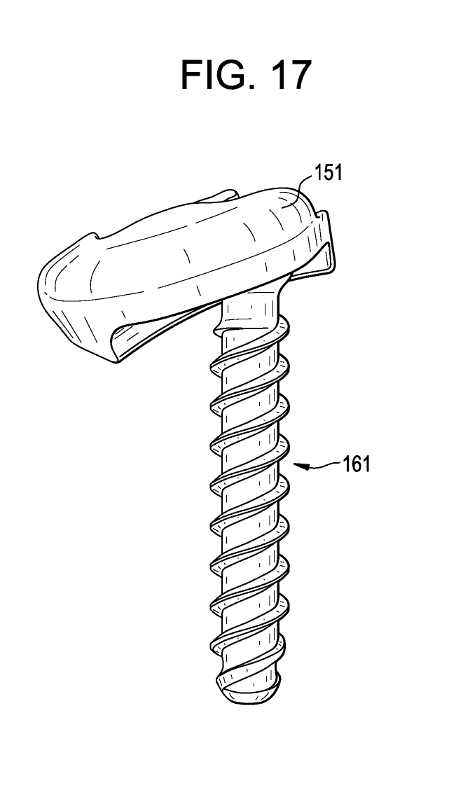

FIGS. 14-17 disclose an embodiment in which the bone posts are eliminated in favor of a single snap-on clip.

Now referring to FIGS. 14-17, there is provided a bone fixation system comprising:
a) a first bone plate 151 having a base 152 and comprising:
   i) a first screw through hole 153 having a first threadform (not shown),
   ii) a second screw throughhole 154 having a second threadform (not shown),
   iii) a peripheral wall 155 extending from the base,
b) a first bone screw 161 having a head 163 and a shaft 165 having a thread 167 thereon that mates with the first threadform;
c) a second bone screw 171 having a head 173 and a shaft 175 having a thread 177 thereon that mates with the second threadform;
d) a snap-on plate 181 having a lip 183 extending therefrom,
wherein the first screw is received in the first screw through hole and extends therethrough,
wherein the second screw is received in the second screw through hole and extends therethrough,
wherein the snap-on plate bears against the peripheral wall and the lip bears against each head.

The utility of the design on FIGS. 14-17 is that the insertion procedure is simplified because it has no locking member that must be rotated. The two screws are automatically locked upon insertion of the snap-on plate.

Now referring back to FIGS. 1-4, there is provided a bone fixation system comprising:
a) a first bone plate 1 having a thickness and comprising:
   i) a bone screw through hole 3 having a first threadform 5,
   ii) a bone anchor through hole 7 having a second threadform 9,
b) a first bone screw having a head 13 and a shaft having a thread thereon that mates with the first threadform;
c) a first bone anchor 19 having a proximal head, an intermediate shaft and a distal tip, the shaft having a proximal threaded portion that mates with the second threadform,
wherein the first screw is received in the first screw through hole and extends therethrough,
wherein the first bone anchor is received in the first bone anchor through hole and extends therethrough,
wherein the head of the bone anchor bears against the head of the bone screw,
wherein the second threadform is more fine than the first threadform.

In this embodiment, the camming of the head of the bone anchor upon the head of the screw acts to prevent screw backout, while the second threadform acts as a means of securing the bone anchor in the bone. The relatively fine nature of this second threadform provides an additional advantage to its backout prevention function by providing high torque locking

In some embodiments, each first threadform (of the bone screw holes) extends for less than one revolution.

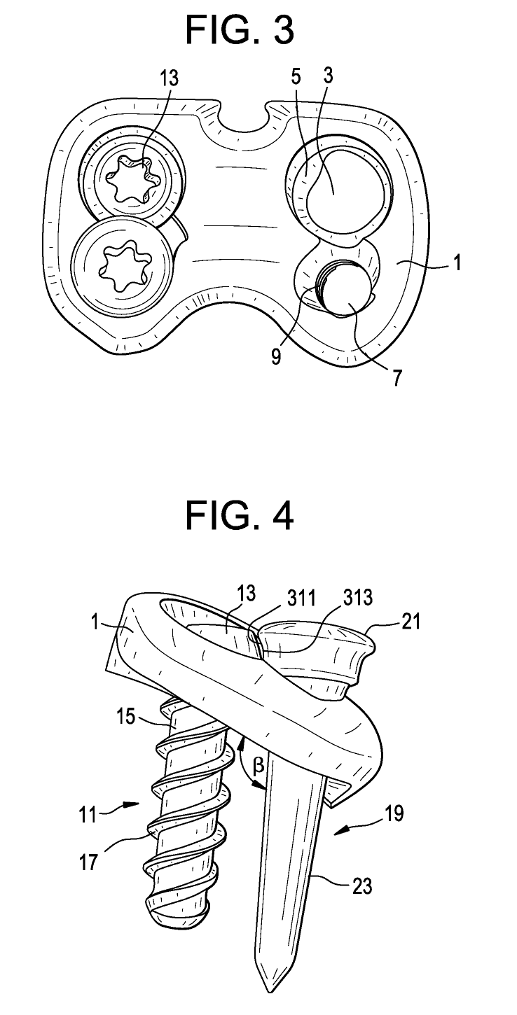

In some embodiments, each screw through hole and its respective post hole overlap, as in FIG. 3. This allows the head of the post to contact the screw head so as to provide a cam lock.

In some embodiments, at least a portion 303 of the plate surrounding each post hole is convex, as in FIG. 2. This topography corresponds to the head of the post.

In some embodiments, the second threadform (which mates with the post) is more fine than the first threadform (which mates with the screw), as in FIG. 3. That is, it comprises more revolutions per unit length. The advantage of this embodiment is that it provides high torque locking

In some embodiments, the bone-contacting surfaces of a bone plate can comprise barbs 301 extending therefrom, as in FIG. 11. These barbs enhance the security of the bone plate to the bone, so that the bone plate acts like a staple.

In some embodiments comprising a single plate, the plate comprises an intermediate portion 201 having a concave surface 203 defining a narrow width, as in FIG. 2. The advantage of the narrowed width of the intermediate portion is its greater ease of delivery through an MIS portal.

In some embodiments comprising a single plate, the plate comprises an intermediate portion 201 whose inner surface 205 is recessed so as to not contact bone, as in FIG. 5. The advantage of this recessed intermediate portion is that it provides for disc and cage clearance, and allows for bone spurs. Accordingly, the plate only touches the patient's anatomy where necessary.

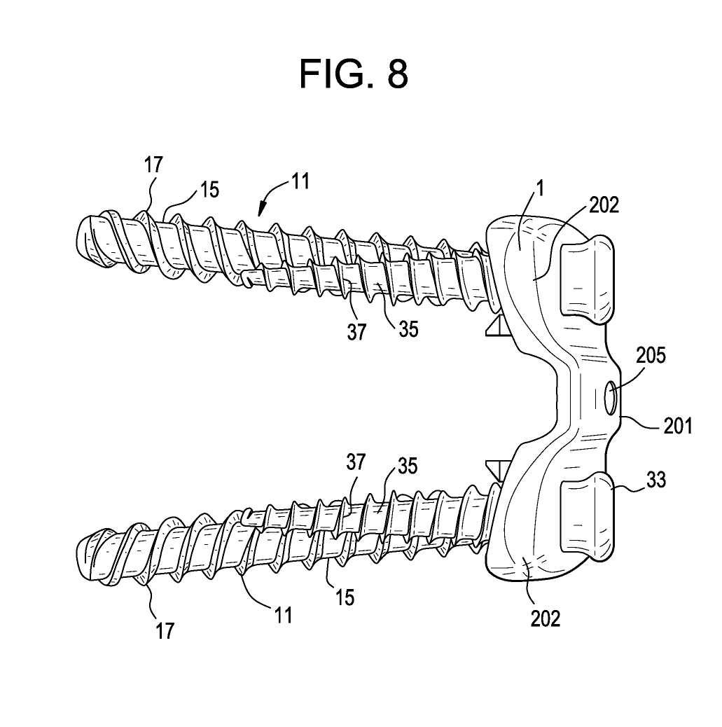

In some embodiments comprising a single plate, the plate comprises a pair of endportions 202 flanking an intermediate portion 201, wherein the thickness of each end portion narrows towards the intermediate portion, as in FIG. 8. The advantage of these narrowing thickness endportions is that it provides for increasing thickness around the bone anchors, thereby providing increased strength around the locking threads.

In some embodiments comprising a single plate, the plate comprises an additional central through hole 205 located in the intermediate portion of the plate, as in FIG. 8. The purpose of this central throughhole is to provide a holding feature for the plate and cage insertion technique. The central throughhole may also be in the form of a threaded hole or divot.

In some embodiments, as in FIG. 4, the head of each screw has a convex surface 311, the head of each post has a mating concave surface 313, and each concave surface bears against its respective convex surface.

In some embodiments, the head of each screw has a concave surface, the head of each post has a mating convex surface, and each convex surface bears against its respective concave surface.

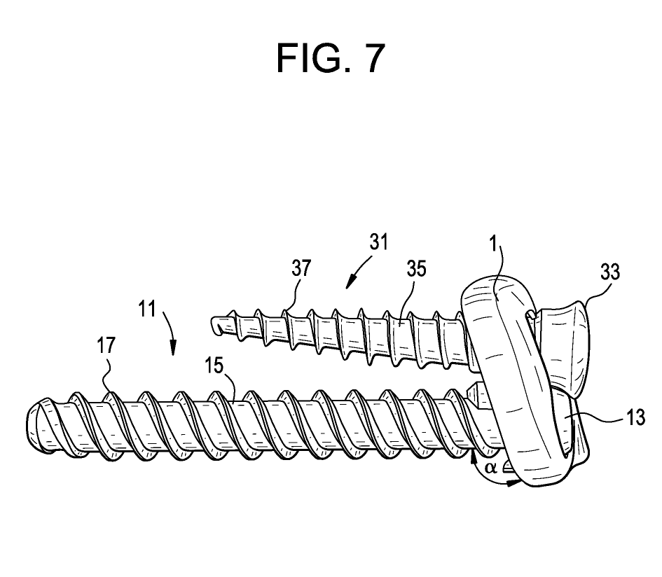

In some embodiments, the screw extends through the plate at a non-orthogonal angle α, as in FIG. 7. The advantage of this non-orthogonal angle is that it provides for maximum bone purchase when the plate becomes parallel across the endplate. Preferably, angle α is greater than 90 degrees.

In some embodiments such as FIG. 5, the system comprises divergent screws. These screws provide a greater bony purchase.

In some embodiments, the shaft of each post is tapered, as in FIG. 4. The tapering has the effect of allowing for easier insertion of the post.

In some embodiments, at least 50% of the shaft of each post is unthreaded, as in FIG. 4. In this condition, the risk of thread-driven backout is mitigated.

In some embodiments, the axial length of each post's thread is substantially the same as the plate thickness.

In some embodiments, the unthreaded portion of the shaft of the post has a feature adapted to promote bone securement. In some embodiments, the feature is a coating adapted to promote tissue ingrowth. In some embodiments, the coating is a hydroxyapatite coating. In some embodiments, the feature is porosity throughout the bulk of the post. In some embodiments, the feature is an osteoconductive layer. In some embodiments, the feature is an expandable post.

In some embodiments, the post (or, more generally, the camming anchor) extends from the plate at a non-orthogonal angle β, as in FIG. 4. The advantage of this non-orthogonal angle is that it helps ergonomics and purchase. In some embodiments, the shaft of each bone post extends from the plate at an angle β that is greater than 90 degrees, and is preferably between 120 degrees and 150 degrees.

In some embodiments, the head of the post (which acts as a cam) can be lobular.

### Operative Technique for Single Plate Version

After access to the affected spinal level is achieved and the appropriate interbody surgery is performed, the lateral/antero-lateral surfaces of adjacent vertebral bodies are prepared for the placement of the plate and anchors. For optimal plate application, the proximal bone contours intimately match the abutting distal plate surfaces. As a general rule, aggressive contouring of the bone or osteophyte debridement may potentially weaken the integrity of the superior and inferior anterior vertebral body cortical rims. Intact cortical rim structure is important for optimal plate positioning and optimal screw purchase. The preferred plate embodiment will generally match the natural contours of the bone rather than require the bone to be machined to match the plate.

Plates are available in a range of 15 to 40 mm and the appropriate matching plate is chosen. An inserter instrument is used to maneuver the plate into the optimum position and the surgeon holds the plate firmly against the vertebral bodies. The bone holes for the first pair of anchors are prepared through the plate holes by awling, drilling, and tapping (or if self drilling/tapping anchors are used, then they are merely awled)). The anchors are then inserted to definitively attach the plate to the bone. The holes for the second pair of anchors/posts are prepared and the second anchors/posts are inserted. The final rotations of the posts are performed with a torque limiting/measuring instrument and the posts are tightened to the appropriate level or until the orientation of a cam/lobe on the second anchor/post is at the desired angular interference position.

### Operative Technique for Triple Plate Version

Two appropriate length bone screws are chosen and snapped into the spherical recess of respective plates. Bone holes are prepared in adjacent vertebral bodies of the spine. The short plates with screws are placed on a self retaining screw driver and threaded/tightened into the bone holes while assuring that the plates are oriented generally parallel to the vertebral body endplates. The plates are firmly lagged to the bones. A third (connector) plate, oriented generally parallel to a cranial-caudal direction, is held in place so that its' superior and inferior adjustment slots are directly over machined thread holes of the small plates. A pair of anchors/posts are introduced through the slots and into the holes such that the third plate is approximated to the two shorter plates. The anchors/posts are tightened and the machined threads are locked with high torque. The approximation of the third plate blocks the spherical recess snap of the two shorter plates resisting back out motion of the bone screws.

We claim:

1. A bone fixation system comprising:
a) a bone plate comprising:
ii) a first screw through hole having a first threadform,
iii) a first post through hole having a second threadform having an axial length,
b) a first bone screw comprising a head and a shaft having a thread thereon that mates with the first threadform;
c) a first bone post comprising a head and a shaft having a thread thereon that mates with the second threadform and has an axial length;
wherein the first screw is received in the first screw through hole and extends therethrough,
wherein the first post is received in the first post through hole and extends therethrough,
wherein the head of the first post bears against the head of the first screw,
wherein the axial length of the thread of the first post is substantially the same as the axial length of the second threadform of the first post through hole.

2. The system of claim 1 wherein the axial length of the first post thread is substantially the same as the plate thickness.

3. The system of claim 2 wherein each first threadform extends for less than one revolution.

4. The system of claim 1 wherein the first screw through hole and the first post hole overlap.

5. The system of claim 1 wherein the head of the first bone screw has a convex surface, the head of the first post has a mating concave surface, and the concave surface bears against the convex surface.

6. The system of claim 1 wherein the head of the first bone screw has a concave surface, the head of the first post has a mating convex surface, and the convex surface bears against the concave surface.

7. The system of claim 1 wherein the shaft of the first post is tapered.

8. The system of claim 1 wherein at least 50% of the length of the shaft of the first post is unthreaded.

9. The system of claim 1 wherein at least a portion of the plate surrounding the first post hole is convex.

10. The system of claim 1 wherein the plate comprises a plurality of barbs to form a staple.

11. The system of claim 1 wherein the bone plate further comprises:
iii) a second screw through hole having the first threadform,
iv) a second post through hole having the second threadform having an axial length,
wherein the system further comprises:
d) a second bone screw having a head and a shaft having a thread thereon that mates with the first threadform;
e) a second bone post having a head and a shaft having a thread thereon that mates with the second threadform and has an axial length;
wherein the second screw is received in the second screw through hole and extends therethrough,
wherein the second post is received in the second post through hole and extends therethrough,
wherein the head of the second post bears against the head of the second screw,
wherein the axial length of the second post thread is substantially the same as the axial length of the second threadform.

12. A bone fixation system comprising:
a) a first bone plate comprising:
i) a first screw through hole having a first threadform,
ii) a first post through hole having a second threadform having an axial length,
iii) a first lip extending therefrom and
b) a first bone screw having a head and a shaft having a thread thereon that mates with the first threadform;
c) a first bone post having a head and a shaft having a thread thereon that mates with the second threadform and has an axial length;
d) a connector plate having a first throughhole,
wherein the first screw is received in the first screw through hole and extends therethrough,
wherein the first post is received in the first post through hole and the connector plate through hole, and extends therethrough,
wherein the connector plate is located between the head of the first post and the first bone plate,
wherein the first lip of the first bone plate bears against the head of the first screw,
wherein the connector plate bears against the first lip,
wherein the axial length of the thread of the first post is substantially the same as the axial length of the second threadform.

13. The system of claim 12 wherein the through hole of the connector plate is a slot.

14. The system of claim 12 wherein the axial length of the first post thread is substantially the same as the first bone plate thickness.

15. The system of claim 12 wherein the first threadform extends for less than one revolution.

16. The system of claim 12 wherein the first screw through hole and the first post hole overlap.

17. The system of claim 12 wherein the head of the first bone screw has a convex surface, the head of the first post has a mating concave surface, and the concave surface bears against the convex surface.

18. The system of claim 12 wherein the head of the first bone screw has a concave surface, the head of the first post has a mating convex surface, and the convex surface bears against the concave surface.

19. The system of claim 12 wherein the shaft of the first post is tapered.

20. The system of claim 12 wherein at least 50% of the shaft of the first post is unthreaded.

21. The system of claim 12 wherein the plate comprises a plurality of barbs to form a staple.

22. The system of claim 12 further comprising:
e) a second bone plate having a thickness and comprising:
i) a second screw through hole having a third threadform,
ii) a second post through hole having a fourth threadform having an axial length,
f) a second bone screw having a head and a shaft having a thread thereon that mates with the third threadform;
g) a second bone post having a head and a shaft having a thread thereon that mates with the fourth threadform and has an axial length;
wherein the connector plate further comprises a second lip extending therefrom and a second throughhole,
wherein the second screw is received in the second screw through hole and extends therethrough,
wherein the second post is received in the second post through hole and the second connector plate through hole, and extends therethrough,
wherein the connector plate is located between the head of the second post and the second bone plate,
wherein the second lip of the connector plate bears against the head of the second screw,
wherein the axial length of the second post thread is substantially the same as the axial length of the fourth threadform.

23. A bone fixation system comprising:
a) a bone plate having a substantially planar bone-contacting surface and comprising:
i) a first through hole having a first threadform,
ii) a second through hole having a second threadform,
b) a first bone anchor having a head and a shaft having a thread thereon that mates with the first threadform;
c) a second bone anchor having a head and a shaft having a thread thereon that mates with the second threadform;
wherein the first bone anchor is received in the first through hole and extends therethrough,
wherein the second bone anchor is received in the second through hole and extends therethrough,
wherein the head of the first bone anchor bears against the head of the second bone anchor, wherein the shaft of at least one of the bone anchors extends from the plate at an angle that is greater than 90 degrees.

24. The system of claim 23 wherein the shaft of each bone anchor extends from the plate at an angle β that is greater than 90 degrees.

25. The system of claim 23 wherein the shaft of each bone anchor extends from the plate at an angle β that is between 120 degrees and 150 degrees.

26. The system of claim 22 wherein each bone anchor is a bone post.

27. A bone fixation system comprising:
a) a first bone plate comprising:
i) a first screw through hole having a first threadform,
ii) a first post through hole having a second threadform having an axial length,
b) a first bone screw having a head and a shaft having a thread thereon that mates with the first threadform;
c) a first anchor having a head and a shaft having a thread thereon that mates with the second threadform and has an axial length;
d) a connector plate having a first lip extending therefrom and a first throughhole and a second lip extending therefrom and a second throughhole,
e) a second bone plate having a thickness and comprising:
i) a second screw through hole having a third threadform,
ii) a second anchor recess having a fourth threadform having an axial length,
f) a second bone screw having a head and a shaft having a thread thereon that mates with the third threadform;
g) a second anchor having a head and a shaft having a thread thereon that mates with the fourth threadform and has an axial length;
wherein the first screw is received in the first screw through hole and extends therethrough,
wherein the first anchor is received in the first anchor recess,
wherein the connector plate is located between the head of the first anchor and the first bone plate,
wherein the first lip of the connector plate bears against the head of the first screw,
wherein the axial length of the first anchor thread is substantially the same as the axial length of the second threadform,
wherein the second screw is received in the second screw through hole and extends therethrough,
wherein the second anchor is received in the second anchor recess,
wherein the connector plate is located between the head of the second post and the second bone plate,
wherein the second lip of the connector plate bears against the head of the second screw,
wherein the axial length of the second anchor thread is substantially the same as the axial length of the fourth threadform,

28. A bone fixation system comprising:
a) a first bone plate comprising:
i) a first screw through hole having a first threadform,
ii) a first post through hole having a second threadform having an axial length,
b) a first bone screw having a head and a shaft having a thread thereon that mates with the first threadform;
c) a first anchor having a head and a shaft having a thread thereon that mates with the second threadform and has an axial length;
d) a connector plate having a first throughhole and a second throughhole,
e) a second bone plate comprising:
i) a second screw through hole having a third threadform,
ii) a second anchor recess having a fourth threadform having an axial length,
f) a second bone screw having a head and a shaft having a thread thereon that mates with the third threadform;
g) a second anchor having a head and a shaft having a thread thereon that mates with the fourth threadform and has an axial length;
wherein the first screw is received in the first screw through hole and extends therethrough,
wherein the first anchor is received in the first anchor recess,
wherein the connector plate is located between the head of the first anchor and the first bone plate,
wherein the head of the first anchor bears against the head of the first screw,
wherein the axial length of the first anchor thread is substantially the same as the axial length of the second threadform.
wherein the second screw is received in the second screw through hole and extends therethrough,
wherein the second anchor is received in the second anchor recess,
wherein the connector plate is located between the head of the second post and the second bone plate,
wherein the head of the second anchor bears against the head of the second screw,
wherein the axial length of the second anchor thread is substantially the same as the axial length of the fourth threadform.

29. A bone fixation system comprising:
a) a first bone plate comprising:
i) a first screw through hole having a first threadform,
ii) a first post through hole having a second threadform having an axial length,
b) a first bone screw having a head and a shaft having a thread thereon that mates with the first threadform;
c) a first bone post having a proximal head, an intermediate shaft and a distal tip, the shaft having:
i) a proximal threaded portion that mates with the second threadform and has an axial length, and
ii) a distal unthreaded portion;
wherein the first screw is received in the first screw through hole and extends therethrough,
wherein the shaft of the first post passes partially through the first post through hole so that the distal unthreaded portion of the shaft substantially extends beyond the plate and the proximal threaded portion of the shaft substantially remains within the first post through hole.

30. The system of claim 29 wherein the axial length of the proximal threaded portion of the shaft is substantially the same as the axial length of the second threadform.

31. The system of claim 29 wherein the bone post is adapted to prevent backout of the bone screw from the first screw throughhole.

32. The system of claim 31 wherein the bone post directly prevents backout of the bone screw from the first screw throughhole.

33. The system of claim 31 wherein the bone post indirectly prevents backout of the bone screw from the first screw throughhole.

34. A bone fixation system comprising:
a) a first bone plate having a thickness and comprising:
i) a bone screw through hole having a first threadform,
ii) a bone anchor through hole having a second threadform,
b) a first bone screw having a head and a shaft having a thread thereon that mates with the first threadform;
c) a first bone anchor having a proximal head, an intermediate shaft and a distal tip, the shaft having a proximal threaded portion that mates with the second threadform,
wherein the first screw is received in the first screw through hole and extends therethrough,
wherein the first bone anchor is received in the first bone anchor through hole and extends therethrough,
wherein the head of the bone anchor bears against the head of the bone screw,
wherein the second threadform is more fine than the first threadform.

35. The system of claim 34 wherein the first bone anchor is a post, and the shaft of the post has a distal unthreaded portion.

36. The system of claim 34 wherein the first bone anchor is a screw, and the shaft of the post has a distal threaded portion.

37. The system of claim 34 wherein the shaft of the bone screw is substantially cylindrical and the shaft of the bone anchor is tapered.

38. A bone fixation system comprising:
a) a first bone plate having a base and comprising:
i) a first screw through hole having a first threadform,
ii) a second screw throughhole having a second threadform,
iii) a peripheral wall extending from the base,
b) a first bone screw having a head and a shaft having a thread thereon that mates with the first threadform;
c) a second bone screw having a head and a shaft having a thread thereon that mates with the second threadform;
d) a snap-on plate having a lip extending therefrom,
wherein the first screw is received in the first screw through hole and extends therethrough,
wherein the second screw is received in the second screw through hole and extends therethrough,
wherein the snap-on plate bears against the peripheral wall and the lip bears against each head.
